(19) United States
(12) Patent Application Publication
(10) Pub. No.: US 2026/0174978 A1
(43) Pub. Date: Jun. 25, 2026

(54) AROMATHERAPY HUMIDIFIER WITH LARGE INTERNAL WATER STORAGE SPACE AND HIGH OPERATIONAL SAFETY

(71) Applicant: Huizhou Pinrui Industrial Co., Ltd., Huizhou (CN)

(72) Inventor: DeJi LI, Bazhong (CN)

(73) Assignee: Huizhou Pinrui Industrial Co., Ltd., Huizhou (CN)

(21) Appl. No.: 19/426,005

(22) Filed: Dec. 18, 2025

(30) Foreign Application Priority Data
Dec. 25, 2024 (CN) 2024232146504

(51) Int. Cl. A61M 11/00

(52) U.S. Cl. CPC A61M 11/005; A61M 2202/0007; A61M 2202/04; A61M 2205/05; A61M 2205/103; A61M 2205/50; A61M 2205/583

(57) ABSTRACT

The present disclosure provides an aromatherapy humidifier with a large internal water storage space and high operational safety, including a humidifier main body, an atomizing plate assembly disposed inside the humidifier main body, a turbine fan, and a main control circuit board, a spray nozzle being provided at an upper part of the humidifier main body, and further including a first LED light panel mounted at the upper part of the humidifier main body and disposed near the spray nozzle, where the humidifier main body includes a water storage tank; a blower nozzle communicating with the turbine fan is provided at the center of an upper side of an inner wall of the water storage tank; and the blower nozzle is disposed close to the first LED light panel and the spray nozzle.

### CROSS-REFERENCE TO RELATED APPLICATIONS

The application claims priority to Chinese patent application No. 2024232146504, filed on Dec. 25, 2024, the entire contents of which are incorporated herein by reference.

### TECHNICAL FIELD

The present disclosure relates to the technical field of humidifier products, in particular to an aromatherapy humidifier with a reasonable structural design, an outstanding application effect, a large internal water storage space and high operational safety.

### BACKGROUND

A humidifier is an appliance that uses high-frequency oscillations generated by ultrasonic oscillation equipment to decompose water molecules and dissolved plant essential oils into nanoscale cold mist with a diameter of several micrometers, which is then dispersed into the surrounding air to fill the air with fragrance.

With the continuous advancement of technology and the gradual improvement of products, existing humidifier products have made significant improvements and enhancements and in terms of appearance and functional characteristics. However, there still exists some shortcomings. For example, a light panel column on an inner side of the humidifier is usually disposed in the middle of an inner side of a water tank, and disposed laterally on the inner side of the water tank, and an air outlet is disposed at one end of the water tank. In practical applications, such a design not only results in uneven air output but also makes water in the water tank prone to backflow into the air outlet when the product is slightly tilted by a certain angle, thereby causing damage to components.

For example, the invention patent with the application number of CN202123065415.1 and entitled “FLAME HUMIDIFIER WITH BACKFLOW STRUCTURE” specifically discloses a flame humidifier with a backflow structure. The flame humidifier with the backflow structure includes: a machine shell, the machine shell being provided with a top cover, and an air outlet hole is provided in the top cover; a water tank, the water tank being fixedly provided between the machine shell and the top cover, and an accommodating space being reserved within the water tank to accommodate a liquid; a vibration mechanism, the vibration mechanism being in contact with the liquid in the accommodating space to atomize the liquid to release fragrance; a fan, the fan being provided in the machine shell for conveying mist atomized by the vibration mechanism; and a flow diversion structure, the flow diversion structure being provided at the top cover for guiding condensed water in the flame humidifier to flow into the water tank. The flame humidifier with the backflow structure provided by the present disclosure is simple in structure, and the condensed water formed on an inner wall of the humidifier flows back to the accommodating space through the flow diversion structure to prevent a water seepage phenomenon of the humidifier, thereby improving user experience.

The aforementioned existing design has the problem mentioned above.

### SUMMARY

The problem in the prior art solved by the present application is:

In a luminous humidifier product in the prior art, a light panel column on an inner side of the product is usually disposed in the middle of an inner side of a water tank, and disposed laterally on the inner side of the water tank, and an air outlet is disposed at one end of the water tank. In practical applications, such a design not only results in uneven air output but also makes water in the water tank prone to backflow into the air outlet when the product is slightly tilted by a certain angle, thereby causing damage to components.

The solution adopted by the present disclosure to solve the technical problem is:
An aromatherapy humidifier with a large internal water storage space and high operational safety is provided, including a humidifier main body, an atomizing plate assembly disposed inside the humidifier main body, a turbine fan, and a main control circuit board, a spray nozzle for spraying out mist being provided at an upper part of the humidifier main body, and
further including a first LED light panel mounted at the upper part of the humidifier main body and disposed near the spray nozzle, where
the humidifier main body includes a water storage tank; the atomizing plate assembly is located at the bottom of the water storage tank; a blower nozzle communicating with the turbine fan is provided at the center of an upper side of an inner wall of the water storage tank;
the blower nozzle is disposed close to the first LED light panel and the spray nozzle; air conveyed by the turbine fan is blown out of the blower nozzle, causing mist to be sprayed evenly from the spray nozzle; and
the atomizing plate assembly, the turbine fan, and the first LED light panel are in electrical connection with the main control circuit board.

Preferably, a vent column is integrally formed inside the water storage box and connected close to an inner wall; the blower nozzle is formed at the top of the vent column;
the aromatherapy humidifier further includes a water baffle disposed at the top of the vent column, wherein a rear part of the water baffle is matched and connected to the top of the vent column, and a front part of the water baffle corresponds to a position of the atomizing plate assembly in a vertical direction to block a mist water column generated by the atomizing plate assembly; and
an atomizing through-hole for exposing the atomizing plate assembly is provided at the bottom of the water storage tank.

Preferably, a light panel slot for placing the first LED light panel is provided on a top side of the humidifier main body; the first LED light panel and the light panel slot are elongated;
the spray nozzle is an elongated opening; and
the vent column and the water baffle are located in the middle of the light panel slot.

Preferably, a second LED light panel for emitting light is also disposed at the bottom of an inner side of the humidifier main body near a front; and
the aromatherapy humidifier further includes a light-transmitting panel and/or a decorative panel disposed on the front of the humidifier main body, wherein the light emitted by the second LED light panel passes through the light-transmitting plate and/or the decorative panel and then is emitted out of the body to present a colorful picture.

Preferably, a gear motor and a rotational connecting rod coaxially connected to an output shaft of the gear motor are also disposed inside the humidifier main body;
a reflector plate for reflecting is disposed on the rotational connecting rod; and
the light emitted by the second LED light panel is reflected by the reflector plate and then passes through the light-transmitting plate and/or the decorative panel before exiting out of the body to form a dynamic display effect.

Preferably, a decorative part for enhancing the display effect is also disposed on the front of the humidifier main body; and the decorative part is located at a part between the light-transmitting plate and the decorative panel.

Preferably, the humidifier main body further includes a main housing and a spray cover plate located at an upper part of the main housing; the spray nozzle is provided on the spray cover plate;
a vent hole is provided at the bottom of the main housing; a light-transmitting mounting hole matched with the light-transmitting plate and the decorative panel is provided at the front part of the main housing; and
the second LED light panel is located at the bottom of an inner side of the light-transmitting mounting hole.

Preferably, a limiting step corresponding to the light panel slot in shape and size is provided at an upper part of the water storage tank; and when the water storage tank is mounted in the humidifier main body, the limiting step is overlapped at an upper part of the light panel slot.

Preferably, an atomizing plate locking portion for fixing the atomizing plate assembly is also integrally formed and disposed at the bottom of the water storage tank; and
an air guide channel communicating with the blower nozzle is provided on a side of the water storage tank.

Preferably, a motor fixing column for fixedly disposing the gear motor is disposed inside the humidifier main body; the gear motor and the rotational connecting rod are disposed laterally inside the humidifier main body; and
the lengths of the rotational connecting rod and the reflector plate correspond to the length of the second LED light panel.

The technical effects achieved by the present application in solving the technical problem are as follows:
Compared with the prior art, the present disclosure provides an aromatherapy humidifier with a large internal water storage space and high operational safety, provided with a humidifier main body 11, an atomizing plate assembly 14 disposed inside the humidifier main body 11, a turbine fan, and a main control circuit board 18, a spray nozzle 121 for spraying out mist being provided at an upper part of the humidifier main body 11, and further including a first LED light panel 16 mounted at the upper part of the humidifier main body 11 and disposed near the spray nozzle 121, where the humidifier main body 11 includes a water storage tank 115; the atomizing plate assembly 14 is located at the bottom of the water storage tank 115; a blower nozzle 1154 communicating with the turbine fan is provided at the center of an upper side of an inner wall of the water storage tank 115; and the blower nozzle 1154 is disposed close to the first LED light panel 16 and the spray nozzle 121. Air conveyed by the turbine fan is blown out of the blower nozzle 1154, causing mist to be sprayed evenly from the spray nozzle 121. The atomizing plate assembly 14, the turbine fan, and the first LED light panel 16 are in electrical connection with the main control circuit board 18. In practical applications, this can achieve a good mist and light display effect, and water inside the water storage tank 115 is less likely to backflow into a functional area when the body is tilted, thereby enhancing the operational safety of the product.

### BRIEF DESCRIPTION OF DRAWINGS

FIG. 1 and FIG. 2 are schematic diagrams of a three-dimensional state structure of an aromatherapy humidifier with a large internal water storage space and high operational safety according to the present disclosure;

FIG. 3 is a schematic diagram of an explosion state structure of an aromatherapy humidifier with a large internal water storage space and high operational safety according to the present disclosure; and

FIG. 4 and FIG. 5 are schematic diagrams of a three-dimensional state structure of a water storage box in an aromatherapy humidifier with a large internal water storage space and high operational safety according to the present disclosure.

In the figures:
Humidifier main body 11; Control button 111; Decorative panel 112; Vent hole 113; Main housing 114; Water storage tank 115; Atomizing through-hole 1151; Vent column 1152; Limiting step 1153; Blower nozzle 1154; Air guide channel 1155; Atomizing plate locking portion 1156; Spray cover plate 12; Spray nozzle 121; Water baffle 13; Atomizing plate assembly 14; Gear motor 15; First LED light panel 16; Second LED light panel 17; Main control circuit board 18; Light-transmitting plate 19; Decorative part 191.

### DETAILED DESCRIPTION OF THE EMBODIMENTS

To make the objectives, technical solutions, and advantages of the present disclosure clearer, the present disclosure will be further described in detail below with reference to the accompanying drawings and embodiments.

It should be understood that the specific embodiments described herein are merely for explaining the present disclosure and are not intended to limit the present disclosure.

Based on the embodiments of the present disclosure, all other embodiments obtained by those skilled in the art without creative effort are within the scope of protection of the present disclosure.

It should be noted that when a component is referred to as being “fixed to” another component, it can be directly fixed on another component, or there may exist an intermediate component therebetween. When a component is considered to be “connected” to another component, it can be directly connected to another component, or there may exist an intermediate component therebetween at the same time.

Unless otherwise defined, all technical and scientific terms used herein have the same meaning as commonly understood by one of ordinary skill in the art to which the present disclosure pertains.

The terminologies used in this specification of the present disclosure herein are for the purpose of describing particular embodiments only and are not intended to limit the present disclosure. The terminology “and/or” as used herein includes any and all combinations of one or more of the related listed items.

Some embodiments of the present disclosure are described in detail below with reference to the accompanying drawings. In the case of no conflict, the following embodiments and features in the embodiments may be combined with each other.

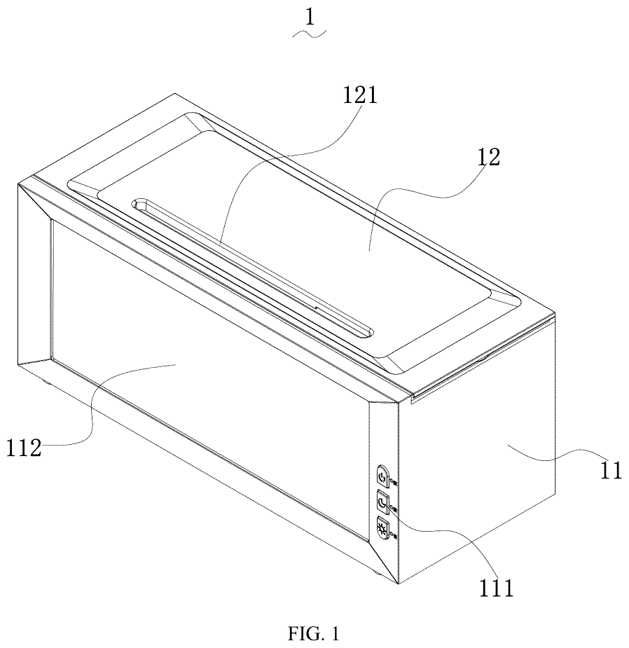

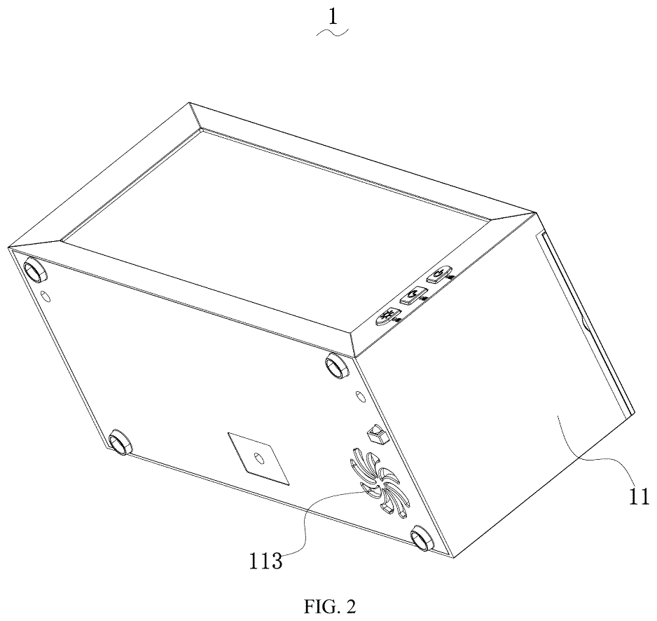

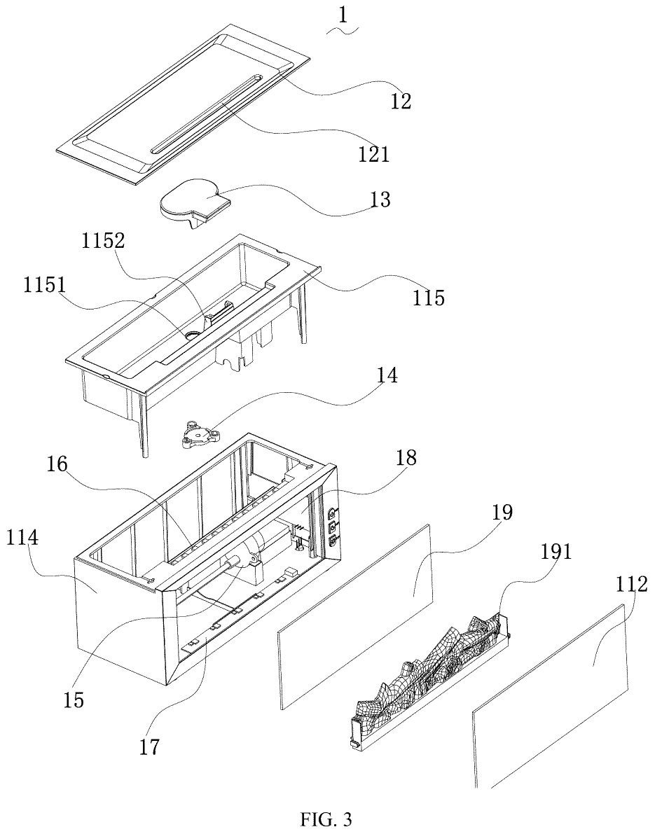

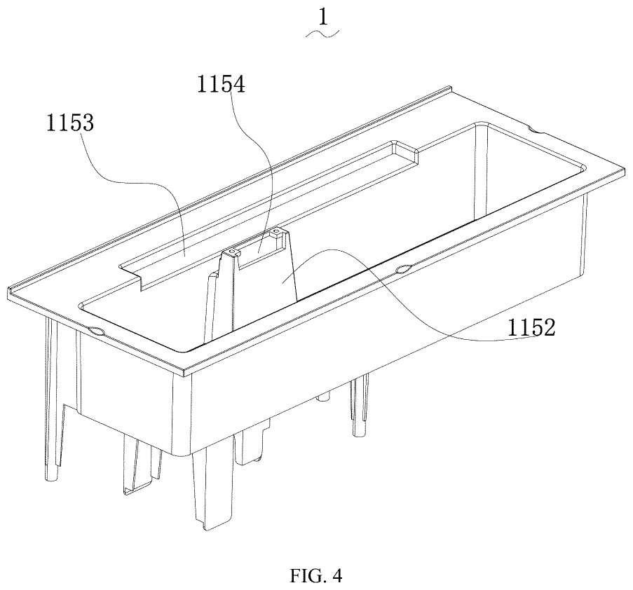

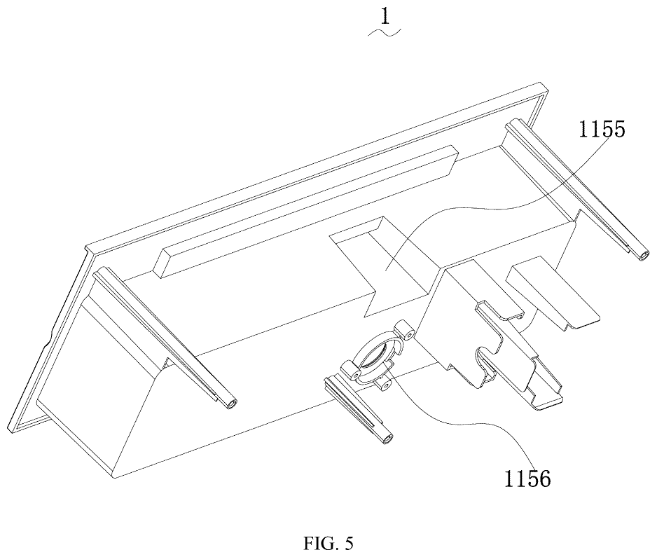

Please refer to FIG. 1 to FIG. 5, the present disclosure provides an aromatherapy humidifier 1 with a large internal water storage space and high operational safety, including a humidifier main body 11, an atomizing plate assembly 14 disposed inside the humidifier main body 11, a turbine fan, and a main control circuit board 18, a spray nozzle 121 for spraying out mist being provided at an upper part of the humidifier main body 11, and the spray nozzle 121 being allowed to be a plurality of round holes or elongated through-holes, etc.;

further including a first LED light panel 16 mounted at the upper part of the humidifier main body 11 and disposed near the spray nozzle 121, wherein in a specific design, the upper part of the humidifier main body 11, especially the spray nozzle 121 and the first LED light panel 16, can be set as a transparent or semi-transparent structure for light to be emitted, which, combined with the mist, forms a visual effect of smoke and clouds;
the humidifier main body 11 includes a water storage tank 115; the atomizing plate assembly 14 is located at the bottom of the water storage tank 115; a blower nozzle 1154 communicating with the turbine fan is provided at the center of an upper side of an inner wall of the water storage tank 115;
the blower nozzle 1154 is disposed close to the first LED light panel 16 and the spray nozzle 121; air conveyed by the turbine fan is blown out of the blower nozzle 1154, causing mist to be sprayed evenly from the spray nozzle 121; and
the atomizing plate assembly 14, the turbine fan, and the first LED light panel 16 are in electrical connection with the main control circuit board 18.

The present disclosure is provided with a humidifier main body 11, an atomizing plate assembly 14 disposed inside the humidifier main body 11, a turbine fan, and a main control circuit board 18, a spray nozzle 121 for spraying out mist being provided at an upper part of the humidifier main body 11, and further including a first LED light panel 16 mounted at the upper part of the humidifier main body 11 and disposed near the spray nozzle 121, where the humidifier main body 11 includes a water storage tank 115; the atomizing plate assembly 14 is located at the bottom of the water storage tank 115; a blower nozzle 1154 communicating with the turbine fan is provided at the center of an upper side of an inner wall of the water storage tank 115; and the blower nozzle 1154 is disposed close to the first LED light panel 16 and the spray nozzle 121. Air conveyed by the turbine fan is blown out of the blower nozzle 1154, causing mist to be sprayed evenly from the spray nozzle 121. The atomizing plate assembly 14, the turbine fan, and the first LED light panel 16 are in electrical connection with the main control circuit board 18. In practical applications, this can achieve a good mist and light display effect, and water inside the water storage tank 115 is less likely to backflow into a functional area when the body is tilted, thereby enhancing the operational safety of the product.

In some other embodiments, a vent column 1152 is integrally formed inside the water storage box 115 and connected close to an inner wall; the blower nozzle 1154 is formed at the top of the vent column 1152;
since the vent column 1152 is disposed close to the inner wall, compared with the prior art, the cuboid light panel column in the middle is eliminated, which may make better use of the internal water storage space of the water storage tank 115;
the aromatherapy humidifier further includes a water baffle 13 disposed at the top of the vent column 1152, where a rear part of the water baffle 13 is matched and connected to the top of the vent column 1152, and a front part of the water baffle 13 corresponds to a position of the atomizing plate assembly 14 in a vertical direction to block a mist water column generated by the atomizing plate assembly 14; the air can also be more evenly outputted from the spray nozzle 121; and
an atomizing through-hole 1151 for exposing the atomizing plate assembly 14 is provided at the bottom of the water storage tank 115.

A light panel slot for placing the first LED light panel 16 is provided on a top side of the humidifier main body 11; the first LED light panel 16 and the light panel slot are elongated;
the spray nozzle 121 is an elongated opening; and
the vent column 1152 and the water baffle 13 are located in the middle of the light panel slot.

A second LED light panel 17 for emitting light is also disposed at the bottom of an inner side of the humidifier main body 11 near a front; and
the aromatherapy humidifier further includes a light-transmitting panel 19 and/or a decorative panel 112 disposed on the front of the humidifier main body 11, where the light emitted by the second LED light panel 17 passes through the light-transmitting plate 19 and/or the decorative panel 112 and then is emitted out of the body to present a colorful picture.

A gear motor 15 and a rotational connecting rod coaxially connected to an output shaft of the gear motor 15 are also disposed inside the humidifier main body 11;
a reflector plate for reflecting is disposed on the rotational connecting rod;
the reflector plate may be designed to have structural features such as a corrugated shape, so as to facilitate the emission of irregular reflected light and create a combustion effect similar to flame, etc.; and
the light emitted by the second LED light panel 17 is reflected by the reflector plate and then passes through the light-transmitting plate 19 and/or the decorative panel 112 before exiting out of the body to form a dynamic display effect.

A decorative part 191, such as a rockery, for enhancing the display effect is also disposed on the front of the humidifier main body 11; and the decorative part 191 is located at a part between the light-transmitting plate 19 and the decorative panel 112.

The humidifier main body 11 further includes a main housing 114 and a spray cover plate 12 located at an upper part of the main housing 114; the spray nozzle 121 is provided on the spray cover plate 12;
a vent hole 113 is provided at the bottom of the main housing 114; a light-transmitting mounting hole matched with the light-transmitting plate 19 and the decorative panel 112 is provided at the front part of the main housing 114;
in the practical design, the turbo fan may be disposed close to the inner side of the vent hole 113, and the external air flows to the air guide channel 1155 after entering the turbo fan; and
the second LED light panel 17 is located at the bottom of an inner side of the light-transmitting mounting hole.

A limiting step 1153 corresponding to the light panel slot in shape and size is provided at an upper part of the water storage tank 114; and when the water storage tank 114 is mounted in the humidifier main body 11, the limiting step 1153 is overlapped at an upper part of the light panel slot.

An atomizing plate locking portion 1156 for fixing the atomizing plate assembly 14 is also integrally formed and disposed at the bottom of the water storage tank 114; and
an air guide channel 1155 communicating with the blower nozzle 1154 is provided on a side of the water storage tank 115.

A motor fixing column for fixedly disposing the gear motor 15 is disposed inside the humidifier main body 11; the gear motor 15 and the rotational connecting rod are disposed laterally inside the humidifier main body 11; and
the lengths of the rotational connecting rod and the reflector plate correspond to the length of the second LED light panel 17.

The humidifier main body further includes a control button 111 disposed on the front of the humidifier main body 11.

The technical effects achieved by the present application in solving the technical problem are as follows:
Compared with the prior art, the present disclosure provides an aromatherapy humidifier 1 with a large internal water storage space and high operational safety, provided with a humidifier main body 11, an atomizing plate assembly 14 disposed inside the humidifier main body 11, a turbine fan, and a main control circuit board 18, a spray nozzle 121 for spraying out mist being provided at an upper part of the humidifier main body 11, and further including a first LED light panel 16 mounted at the upper part of the humidifier main body 11 and disposed near the spray nozzle 121, where the humidifier main body 11 includes a water storage tank 115; the atomizing plate assembly 14 is located at the bottom of the water storage tank 115; a blower nozzle 1154 communicating with the turbine fan is provided at the center of an upper side of an inner wall of the water storage tank 115; and the blower nozzle 1154 is disposed close to the first LED light panel 16 and the spray nozzle 121. Air conveyed by the turbine fan is blown out of the blower nozzle 1154, causing mist to be sprayed evenly from the spray nozzle 121. The atomizing plate assembly 14, the turbine fan, and the first LED light panel 16 are in electrical connection with the main control circuit board 18. In practical applications, this can achieve a good mist and light display effect, and water inside the water storage tank 115 is less likely to backflow into a functional area when the body is tilted, thereby enhancing the operational safety of the product.

The embodiments of the present disclosure described above do not constitute a limitation on the scope of protection of the present disclosure. Any modifications, equivalent substitutions, improvements, etc. made within the spirit and principles of the present disclosure shall be included within the scope of protection of the claims of the present disclosure.

What is claimed is:

1. An aromatherapy humidifier with a large internal water storage space and high operational safety, comprising a humidifier main body, an atomizing plate assembly disposed inside the humidifier main body, a turbine fan, and a main control circuit board, a spray nozzle being provided at an upper part of the humidifier main body, and
further comprising a first LED light panel mounted at the upper part of the humidifier main body and disposed near the spray nozzle, wherein
the humidifier main body comprises a water storage tank; the atomizing plate assembly is located at the bottom of the water storage tank; a blower nozzle communicating with the turbine fan is provided at the center of an upper side of an inner wall of the water storage tank;
the blower nozzle is disposed close to the first LED light panel and the spray nozzle; air conveyed by the turbine fan is blown out of the blower nozzle, causing mist to be sprayed evenly from the spray nozzle; and
the atomizing plate assembly, the turbine fan, and the first LED light panel are in electrical connection with the main control circuit board.

2. The aromatherapy humidifier with a large internal water storage space and high operational safety according to claim 1, wherein a vent column is integrally formed inside the water storage box and connected close to an inner wall; the blower nozzle is formed at the top of the ventilation column;
the aromatherapy humidifier further comprises a water baffle disposed at the top of the vent column, wherein a rear part of the water baffle is matched and connected to the top of the vent column, and a front part of the water baffle corresponds to a position of the atomizing plate assembly in a vertical direction to block a mist water column generated by the atomizing plate assembly; and
an atomizing through-hole for exposing the atomizing plate assembly is provided at the bottom of the water storage tank.

3. The aromatherapy humidifier with a large internal water storage space and high operational safety according to claim 2, wherein a light panel slot for placing the first LED light panel is provided on a top side of the humidifier main body; the first LED light panel and the light panel slot are elongated;
the spray nozzle is an elongated opening; and
the vent column and the water baffle are located in the middle of the light panel slot.

4. The aromatherapy humidifier with a large internal water storage space and high operational safety according to claim 1, wherein a second LED light panel for emitting light is also disposed at the bottom of an inner side of the humidifier main body near a front; and
the aromatherapy humidifier further comprises a light-transmitting panel and/or a decorative panel disposed on the front of the humidifier main body, wherein the light emitted by the second LED light panel passes through the light-transmitting plate and/or the decorative panel and then is emitted out of the body to present a colorful picture.

5. The aromatherapy humidifier with a large internal water storage space and high operational safety according to claim 4, wherein a gear motor and a rotational connecting rod coaxially connected to an output shaft of the gear motor are also disposed inside the humidifier main body;
a reflector plate for reflecting is disposed on the rotational connecting rod; and
the light emitted by the second LED light panel is reflected by the reflector plate and then passes through the light-transmitting plate and/or the decorative panel before exiting out of the body to form a dynamic display effect.

6. The aromatherapy humidifier with a large internal water storage space and high operational safety according to claim 5, wherein a decorative part for enhancing the display effect is also disposed on the front of the humidifier main body; and the decorative part is located at a part between the light-transmitting plate and the decorative panel.

7. The aromatherapy humidifier with a large internal water storage space and high operational safety according to claim 5, wherein the humidifier main body further comprises a main housing and a spray cover plate located at an upper part of the main housing; the spray nozzle is provided on the spray cover plate;
a vent hole is provided at the bottom of the main housing; a light-transmitting mounting hole matched with the light-transmitting plate and the decorative panel is provided at the front part of the main housing; and
the second LED light panel is located at the bottom of an inner side of the light-transmitting mounting hole.

8. The aromatherapy humidifier with a large internal water storage space and high operational safety according to claim 3, wherein a limiting step corresponding to the light panel slot in shape and size is provided at an upper part of the water storage tank; and when the water storage tank is mounted in the humidifier main body, the limiting step is overlapped at an upper part of the light panel slot.

9. The aromatherapy humidifier with a large internal water storage space and high operational safety according to claim 1, wherein an atomizing plate locking portion for fixing the atomizing plate assembly is also integrally formed and disposed at the bottom of the water storage tank; and
an air guide channel communicating with the blower nozzle is provided on a side of the water storage tank.

10. The aromatherapy humidifier with a large internal water storage space and high operational safety according to claim 5, wherein a motor fixing column for fixedly disposing the gear motor is disposed inside the humidifier main body; the gear motor and the rotational connecting rod are disposed laterally inside the humidifier main body; and
the lengths of the rotational connecting rod and the reflector plate correspond to the length of the second LED light panel.
